(19) United States
(12) Patent Application Publication
(10) Pub. No.: US 2010/0062671 A1
(43) Pub. Date: Mar. 11, 2010

(54) COMPOSITE WIPE

(75) Inventors: William Child, Lock Haven, PA (US); Robin Damaghi, Great Neck, NY (US); Lawrence E. Duane, Wellesley, MA (US)

Correspondence Address: AMSTER, ROTHSTEIN & EBENSTEIN LLP, 90 PARK AVENUE, NEW YORK, NY, 10016, US

(73) Assignee: Nutek Disposables, Inc., McElhattan, PA (US)

(21) Appl. No.: 12/205,478

(22) Filed: Sep. 5, 2008

(51) Int. Cl. A61F 13/515; B32B 5/26; D04H 1/46

(52) U.S. Cl. 442/382; 156/166; 28/104; 264/103

(57) ABSTRACT

A wipe including at least one non-woven web layer of discontinuous fibers; and a spunbond-meltblown-spunbond web layer of continuous fibers positioned in facing and adjacently contacting relation with the at least one non-woven web layer, wherein the wipe has an opacity index of at least 1.3, where the opacity index is calculated based on the following equation:

opacity index=(opacity of the wipe)/(total basis weight of the wipe).

### FIELD OF THE INVENTION

The present invention relates to wipes for sanitary purposes, and in particular to wipes having a multi-layer structure.

### BACKGROUND

Sanitary wipes are well known commercial consumer products that have been used for baby wipes, hand wipes, household cleaning wipes, industrial wipes and the like. Conventional wipes include a single layer of substantially homogenous material. For example, some singe layer wipes have included an air laid web of fibers which are uniformly mixed or distributed throughout the web. Such singe layer wipes have also included polymeric fibers such as polyester, polyethylene and polypropylene and natural fibers or synthetic fibers such as cellulosic fibers.

However, with such single layer wipes, it is difficult to obtain the necessary balance of physical characteristics. In particular, depending on the application, physical characteristics of a wipe, such as softness, flexibility, strength, thickness, texture, integrity, opacity and resiliency, need to be optimized.

### SUMMARY OF THE INVENTION

A wipe according to an exemplary embodiment of the present invention comprises: at least one nonwoven web layer of discontinuous fibers; and a spunbond-meltblown-spunbond web layer of continuous fibers positioned in facing and adjacently contacting relation with the at least one nonwoven web layer wherein the wipe has an opacity index of at least 1.3, where the opacity index is calculated based on the following equation:

opacity index=(opacity of the wipe)/(total basis weight of the wipe).

In at least one embodiment, the at least one nonwoven web layer comprises a first nonwoven web layer and a second nonwoven web layer, the spunbond-meltblown-spunbond web layer disposed between the first and second nonwoven web layers.

In at least one embodiment, the discontinuous fibers comprise rayon fibers, natural fibers and polymeric fibers.

In at least one embodiment, the natural fibers comprise at least one of the following natural fiber types: cotton, bamboo, hemp, polylactide and pulp.

In at least one embodiment, the polymeric fibers comprise at least one of the following polymeric fiber types: polypropylene and polyester.

In at least one embodiment, the at least one nonwoven web layer is one of the following types of web layers: carded fiber web layer, air-laid fiber web layer and wet-laid fiber web layer.

In at least one embodiment, the at least one nonwoven web layer has a basis of weight within the range of approximately 5 gsm to approximately 55 gsm.

In at least one embodiment, the spunbond-meltblown-spunbond web layer comprises polypropylene.

In at least one embodiment, the spunbond-meltblown-spunbond web layer comprises polylactide.

In at least one embodiment, the spunbond-meltblown-spunbond web layer is unbonded.

In at least one embodiment, the spunbond-meltblown-spunbond web layer is bonded.

In at least one embodiment, the spunbond-meltblown-spunbond web layer has a basis of weight within the range of approximately 5 gsm to approximately 35 gsm.

In at least one embodiment, the at least one nonwoven web layer is bonded with the spunbond-meltblown-spunbond material layer.

In at least one embodiment, the wipe further comprises a liquid.

In at least one embodiment, the total basis weight of the wipe is at least 20 gsm.

In at least one embodiment, the opacity of the wipe is at least 40%.

In at least one embodiment, the ratio of tensile strength in the machine direction of the wipe relative to tensile strength in the cross direction of the wipe is within the range of approximately 2.0 to approximately 3.0.

In at least one embodiment, the ratio of percentage elongation in the cross direction of the wipe relative to percentage elongation in the machine direction of the wipe is within the range of approximately 1.0 to approximately 1.5.

In at least one embodiment, the wipe has an opacity-cross dimensional tensile strength index of at least 0.5, where the opacity-cross dimensional tensile strength index is calculated based on the following equation:

opacity-cross dimensional tensile strength index=((opacity of the wipe)(cross dimensional tensile strength of the wipe))/(total basis weight of the wipe)².

In at least one embodiment, the wipe has a combination index of at least 0.7, where the combination index is calculated based on the following equation:

combination index=[((opacity of the wipe)(cross dimensional tensile strength of the wipe)(1/cross dimensional elongation of the wipe))/(total basis weight of the wipe)³](10000).

A wipe according to an exemplary embodiment of the present invention comprises: at least one non-woven web layer of discontinuous fibers; and a spunbond-meltblown-spunbond web layer of continuous fibers positioned in facing and adjacently contacting relation with the at least one non-woven web layer, wherein the wipe has a combination index of at least 0.7, where the combination index is calculated based on the following equation:

combination index=[((opacity of the wipe)(cross dimensional tensile strength of the wipe)(1/cross dimensional elongation of the wipe))/(total basis weight of the wipe)³](10000).

A method of forming a wipe according to an exemplary embodiment of the present invention comprises the steps of: forming at least one nonwoven web layer of discontinuous fibers; forming a spunbond-meltblown-spunbond web layer of continuous fibers; and bonding the at least one nonwoven web layer with the spunbond-meltblown-spunbond web layer, wherein the wipe has an opacity index of at least 1.3, where the opacity index is calculated based on the following equation:

opacity index=(opacity of the wipe)/(total basis weight of the wipe).

In at least one embodiment, the step of forming the at least one nonwoven web layer comprises forming a first nonwoven web layer and a second nonwoven web layer.

In at least one embodiment, the method further comprises disposing the spunbond-meltblown-spunbond web layer between the first and second nonwoven web layers.

In at least one embodiment, the step of forming the at least nonwoven web layer comprises using at least one of the following types of web-formation processes: carding, airlaying and wetlaying.

In at least one embodiment, the method further comprises bonding the at least one nonwoven web layer.

In at least one embodiment, the step of bonding the at least one nonwoven web layer comprises using at least one of the following bonding processes: hydroentanglement, thermal bonding, chemical bonding and mechanical bonding.

In at least one embodiment, the method further comprises bonding the spunbond-meltblown-spunbond web layer.

In at least one embodiment, the step of bonding the at least one nonwoven web layer with the spunbond-meltblown-spunbond web layer comprises using at least one of the following bonding processes: hydroentanglement and thermal bonding.

### BRIEF DESCRIPTION OF THE DRAWINGS

The above and related objects, features and advantages of the present invention will be more fully understood by reference to the following, detailed description of the preferred, albeit illustrative, embodiment of the present invention when taken in conjunction with the accompanying figures, wherein:

FIG. 1 is a cross-sectional view of a wipe according to an exemplary embodiment of the present invention;

FIG. 2 is a cross-sectional view of a wipe according to another exemplary embodiment of the present invention; and

FIG. 3 is as flow-chart showing a method of forming a wipe according to an exemplary embodiment of the present invention.

FIG. 4 is a chart showing opacity versus basis weight for wipes according to various exemplary embodiments of the present invention and comparative examples;

FIG. 5 is a chart showing tensile strength ratio versus basis weight for wipes according to various exemplary embodiments of the present invention and comparative examples;

FIG. 6 is a chart showing elongation ratio versus basis weight for wipes according to various exemplary embodiments of the present invention and comparative examples;

FIG. 7 is a chart showing opacity-cross dimensional tensile strength index versus basis weight for wipes according to various exemplary embodiments of the present invention and comparative examples; and

FIG. 8 is a chart showing combination index versus basis weight for wipes according to various exemplary embodiments of the present invention and comparative examples.

### DETAILED DESCRIPTION OF THE PREFERRED EMBODIMENT(S)

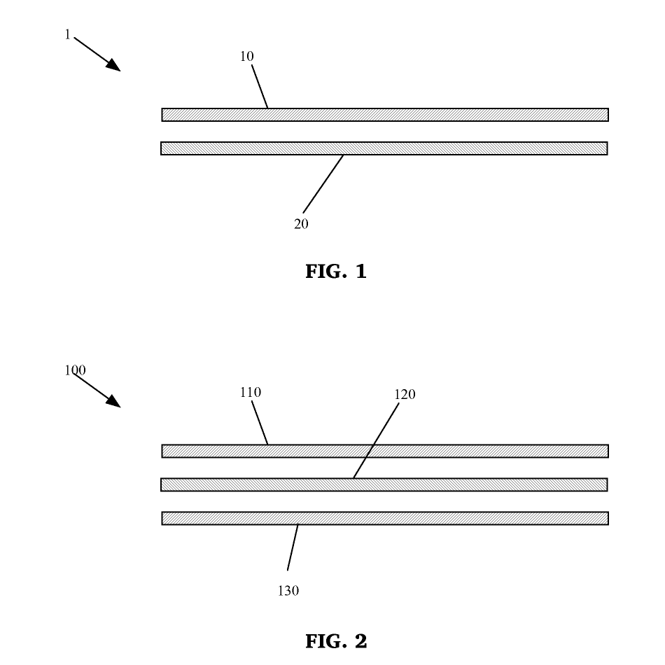

FIG. 1 is a cross-sectional view of a wipe, generally designated by reference number 1, according to an exemplary embodiment of the present invention. The wipe 1 includes a nonwoven web layer 10 and a spunbond-meltblown-spunbond (SMS) web layer 20 positioned in facing and adjacently contacting relation with the nonwoven web layer 10. As explained in further detail below, the SMS web layer 20 provides the wipe 1 with increased opacity, thereby giving the wipe 1 the appearance of a higher weight fabric, without adding significantly to the overall weight of the wipe 1.

The nonwoven web layer 10 is preferably composed of discontinuous fibers of rayon (viscose), in addition to natural discontinuous fibers and polymeric discontinuous fibers. The natural discontinuous fibers used in the nonwoven web layer 10 may be made of, for example, cotton, pulp, bamboo, hemp, or blends of these materials. The polymeric discontinuous fibers used in the nonwoven web layer 10 may be made of, for example, polypropylene or polyester. In another exemplary embodiment, more eco-friendly polymeric materials may be used, such as, for example, polylactide (PLA).

The nonwoven web layer 10 may be formed using any suitable nonwoven process, such as, for example, carding, wetlaying and airlaying processes. The basis weight of the nonwoven web layer 10 is preferably within the range of approximately 5 gsm to approximately 55 gsm. In a preferred embodiment, the nonwoven web layer 10 has a basis weight of 33 gsm. In another preferred embodiment, the nonwoven web layer 10 has a basis weight of 55 gsm.

The SMS web layer 20 is preferably composed of continuous fibers of polymeric material. The polymeric material may be, for example, polyolefins such as polypropylene and polyethylene, polyamides, and polyesters. In another exemplary embodiment, more eco-friendly polymeric materials may be used, such as, for example, polylactide (PLA).

The SMS web layer 20 may be bonded or unbonded. The SMS web layer 20 may be unbonded in that multiple beams of spunbond and meltblown in combination to create an SMS structure can be installed directly into the wipe production line instead of a roll unwind to introduce a previously formed SMS fabric. If bonded, the SMS web layer 10 may be bonded using any suitable bonding process, such as, for example, thermal bonding, hydroentanglement, chemical bonding and mechanical bonding. The SMS web layer 20 preferably has a basis weight within the range of approximately 5 gsm to 35 gsm. In a preferred embodiment, the SMS web layer 20 has a basis weight of 12 gsm. In another preferred embodiment, the SMS web layer 20 has a basis weight of 13.5 gsm. The basis weight of the SMS web layer 20 is selected so that the overall appearance of the wipe 1 is improved by providing increased opacity, increased fabric thickness and improved whiteness. Further, the inclusion of the SMS web layer 20 improves the tensile strength of the wipe 1, without having to increase the overall basis weight of the wipe 1. The SMS web layer 20 may also include a colorant, such as, for example, TiO₂, to further increase the opacity of the wipe 1. In an exemplary embodiment, the amount of colorant added to the SMS web layer 20 may be as high as approximately 5% by weight. In an exemplary embodiment of the present invention, the SMS web layer 20 is an SMS product commercially available from First Quality Nonwovens, of Hazleton, Pa.

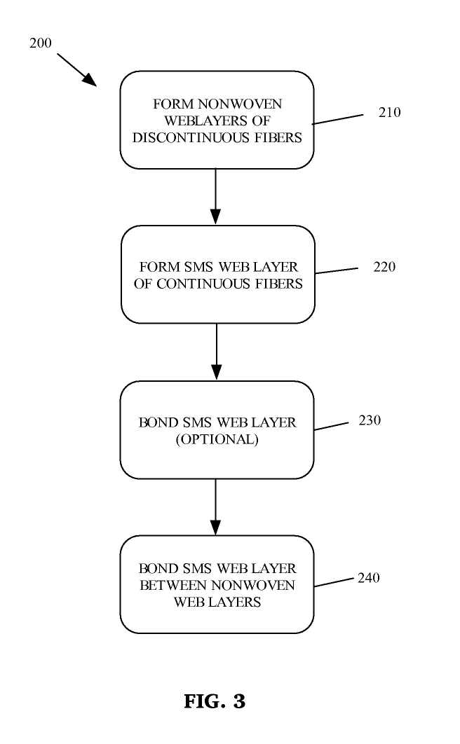

FIG. 2 is a cross-sectional view of a wipe, generally designated by reference number 100, according to another exemplary embodiment of the present invention. The wipe 100 includes a first outer nonwoven web layer 110, a second outer nonwoven web layer 130 and an SMS web layer 120 disposed between the first and second outer nonwoven web layers 110, 130. The first and second nonwoven web layers 110, 130 may have the same structure as the nonwoven web layer 10 described above, including polymeric and natural discontinuous fibers. The SMS web layer 120 may have the same structure as the SMS layer 20 described above, including continuous fibers of polymeric material.

The wipes described herein may also be impregnated with a liquid, so that the wipe becomes a wet wipe. The liquid may be any solution that can be absorbed into or that resides on the wipe, and may include any suitable components that provide the desired wiping properties. For example, the components may include water, emollients, surfactants, fragrances, preservatives, chelating agents, pH buffers, solvents and other cleaning or enhancing agents such as those used in household/industrial applications or combinations thereof as are well known to those skilled in the art. The liquid may also include lotions and/or medicaments.

The wipe described herein may also be subjected to any number and variety of post-processing steps, including, for example, hydro-embossing, thermal embossing, transfer printing (colors or textures) and spray coating.

FIG. 3 is a flowchart showing a method, generally designated by reference number 200, for making a wipe according to an exemplary embodiment of the present invention. In step S210, two nonwoven web layers of discontinuous fibers are formed using any suitable nonwoven process, such as, for example, carding, wetlaying and airlaying processes. The nonwoven web layers may include discontinuous fibers of rayon, in addition to natural discontinuous fibers and polymeric discontinuous fibers. The natural discontinuous fibers used in the nonwoven web layers may be made of, for example, cotton, pulp, bamboo, hemp, or blends of these materials. The polymeric discontinuous fibers used in the nonwoven web layers may be made of, for example, polypropylene or polyester. In another exemplary embodiment, more eco-friendly polymeric materials may be used, such as, for example, polylactide (PLA).

In step S220, a web layer of continuous fibers is formed using a spunbond-meltblown-spunbond process. The continuous fibers may be polymeric material, such as, for example, polyolefins such as polypropylene and polyethylene, polyamides, and polyesters. In an exemplary embodiment, more eco-friendly polymeric materials may be used, such as, for example, polylactide (PLA). In an alternative embodiment, a pre-formed roll of SMS may be provided, where the SMS roll is either bonded or unbonded.

In step S230, the SMS web layer formed in step S220 is subjected to a bonding process. The bonding process may include any suitable bonding process, such as, for example, thermal bonding, hydroentanglement, chemical bonding and mechanical bonding. It should be appreciated that step S230 is optional, and in other exemplary embodiments of the present invention the SMS web layer may be left unbonded.

In step S240, the SMS web layer is bonded between the two nonwoven web layers to form the wipe. The three layers may be bonded together using any suitable bonding process, including, for example, hydroentanglement and thermal bonding.

It should be appreciated that the method of forming the wipe according to the present invention is not limited to the above-described method. For example, in other exemplary embodiments, the bonding of the SMS web layer may take place at the same time as the bonding of the SMS web layer to the nonwoven web layers. Also, the method may include an additional step of impregnating the wipe with fluid, so as to form a wet wipe.

The following examples illustrate the advantages of the present invention:

### Example 0

### E0

A three layer composite was provided. Each outer card web layer had a basis weight of approximately 10 gsm and was made of viscose and polyester, where the blend weight ratio was 50/50, and the inner layer had a 12 gsm SMS structure commercially available from First Quality Nonwovens, of Hazelton, Pa. The total composition was hydroentangled and hydropatterned using a square design. The three layer composite was subjected to the following standard test procedures, which are well known and commonly used in the industry:

### Tensile/Elongation: EDANA: ERT 20.2-89

### Thickness: EDANA: ERT 30.5-99

### Opacity: ASTM: E 1347

### Basis Weight: ASTM: D 6242-98

### Example 1

### E1

A two layer composite was provided. One layer of the composite was a 13 gsm card web made of viscose and polyester, where the viscose to polyester weight ratio was 30/70. The other layer was a 20 gsm SMS structure commercially available from First Quality Nonwovens, of Hazelton, Pa. The total composition was hydroentangled only, and not hydropatterned. The two layer composition was subjected to the same test procedures described in Example 0.

### Example 2

### E2

The same structure as Example 1 was provided, but with a slightly lower basis weight, with the card web weighing 12 gsm. The structure was subjected to the same test procedures described in Example 0.

### Example 3

### E3

The same structure as Example 1 was provided, but with a slightly lower basis weight, with the card web weighing 12 gsm. The structure was subjected to the same test procedures described in Example 0.

### Example 4

### E4

The same structure as Example 1 was provided, but with a slightly lower basis weight, with the card web weighing 10 gsm. The total composite was hydroentangled and hydropatterned using a square design. The structure was subjected to the same test procedures described in Example 0.

### Example 5

### E5

The same structure as Example 1 was provided, but with a slightly lower basis weight, with the card web weighing 10 gsm. The total composite was hydroentangled and not hydropatterned. The structure was subjected to the same test procedures described in Example 0.

### Comparative Example 1

### CE1

A 100% carded web hydroentangled structure was provided. The carded web was made of viscose and polyester, where the viscose to polyester weight ratio was 30/70. The total composition was hydroentangled and hydropatterned using a square design. The structure was subjected to the same test procedures described in Example 0.

### Comparative Example 2

### CE2

A 100% carded web hydroentangled structure was provided. The carded web was made of viscose and polyester, where the viscose to polyester weight ratio was 30/70. This product was not hydropatterned. The structure was subjected to the same test procedures described in Example 0.

### Comparative Example 3

### CE3

A 100% carded web hydroentangled structure was provided. The carded web was made of viscose and polyester, where the viscose to polyester weight ratio was 50/50. The structure was subjected to the same test procedures described in Example 0.

### Comparative Example 4

### CE4

A 100% carded web hydroentangled structure was provided. The carded web was made of viscose and polyester, where the viscose to polyester weight ratio was 30/70. The total composition was hydroentangled and hydropatterned using a square design. The structure was subjected to the same test procedures described in Example 0.

### Comparative Example 5

### CE5

A 100% carded web hydroentangled structure was provided having a lower basis weight than that of Comparative Example 4. The carded web was made of viscose and polyester, where the viscose to polyester weight ratio was 30/70. The total composition was hydroentangled and hydropatterned using a square design. The structure was subjected to the same test procedures described in Example 1.

### Comparative Example 6

### CE6

A 100% carded web hydroentangled structure was provided. The carded web was made of viscose, polypropylene and reclaim fiber, where the blend weight ratio is 29/66/5, respectively. The total composition was hydroentangled and hydropatterned using a square design. The structure was subjected to the same test procedures described in Example 0.

### Comparative Example 7

### CE7

A 100% carded web hydroentangled structure was provided. The carded web was made of viscose and polypropylene, where the viscose to polypropylene weight ratio is 30/70. The total composition was hydroentangled and not hydropatterned. The structure was subjected to the same test procedures described in Example 0.

### Comparative Example 8

### CE8

A 100% carded web hydroentangled structure was provided having a lower basis weight than that of Comparative Example 3. The carded web was made of viscose and polyester, where the viscose to polyester weight ratio was 50/50. The structure was subjected to the same test procedures described in Example 0.

### Comparative Example 9

### CE9

A 100% carded web hydroentangled structure was provided having a higher basis weight than that of Comparative Example 3. The carded web was made of cotton, viscose and polyester, where the cotton, viscose to polyester weight ratio was 15/35/50. The structure was subjected to the same test procedures described in Example 0.

### Comparative Example 10

### CE10

A three-layer composite was provided having outer card web layers and an inner layer of spunbond fabric. Each card web layer had a basis weight of 10 gsm and was made of viscose and polyester having a blend ratio of 50/50. The inner layer had a basis weight of 10 gsm. The total 30 gsm composite was hydroentangled and not hydropatterned.

The results of these tests are provided in Table 1, shown below:

TABLE 1

| | | | | | Wipe Tensile | | | | | |
|---|---|---|---|---|---|---|---|---|---|---|
| | | | | | Strength | | | Elongation | | |
| | Patent | | Opacity | Thickness | (N/5 cm) | | | (%): | | |
| | Code | GSM | (%) | (mm) | MD | CD | MD/CD | MD | CD | CD/MD |
| EXAMPLES | E0 | 31 | 51 | 0.56 | 49 | 19 | 2.5 | 67.2 | 82.9 | 1.2 |
| | E1 | 33 | 55 | 0.50 | 41 | 21 | 1.9 | 64.3 | 76.2 | 1.2 |
| | E2 | 32 | 50 | 0.49 | 41 | 14 | 2.9 | 60.4 | 77.7 | 1.3 |
| | E3 | 32 | 51 | 0.54 | 52 | 20 | 2.7 | 73.4 | 86.7 | 1.2 |
| | E4 | 30 | 51 | 0.55 | 42 | 19 | 2.2 | 73.7 | 82.7 | 1.1 |
| | E5 | 30 | 48 | 0.55 | 52 | 19 | 2.7 | 73.9 | 81.2 | 1.1 |
| COMPARATIVE | CE1 | 31 | 35 | 0.57 | 39 | 9 | 4.5 | 59.4 | 176.0 | 3.0 |
| EXAMPLES | CE2 | 33 | 39 | 0.55 | 54 | 12 | 4.5 | 55.4 | 175.3 | 3.2 |
| | CE3 | 42 | 52 | 0.51 | 94 | 22 | 4.3 | 34.2 | 128.3 | 3.8 |
| | CE4 | 44 | 46 | 0.72 | 74 | 20 | 3.6 | 61.6 | 156.9 | 2.5 |
| | CE5 | 40 | 44 | 0.72 | 63 | 16 | 4.0 | 62.9 | 149.7 | 2.4 |
| | CE6 | 42 | 44 | 0.63 | 79 | 18 | 4.4 | 54.0 | 170.6 | 3.2 |
| | CE7 | 39 | 46 | 0.48 | 49 | 10 | 5.0 | 41.4 | 161.6 | 3.9 |
| | CE8 | 44 | 55 | 0.40 | 79 | 18 | 4.4 | 20.9 | 90.5 | 4.3 |
| | CE9 | 53 | 57 | 0.66 | 97 | 35 | 2.8 | 39.9 | 106.5 | 2.7 |
| | CE10 | 32 | 35 | 0.41 | 40 | 18 | 2.2 | 46.4 | 120.9 | 2.6 |

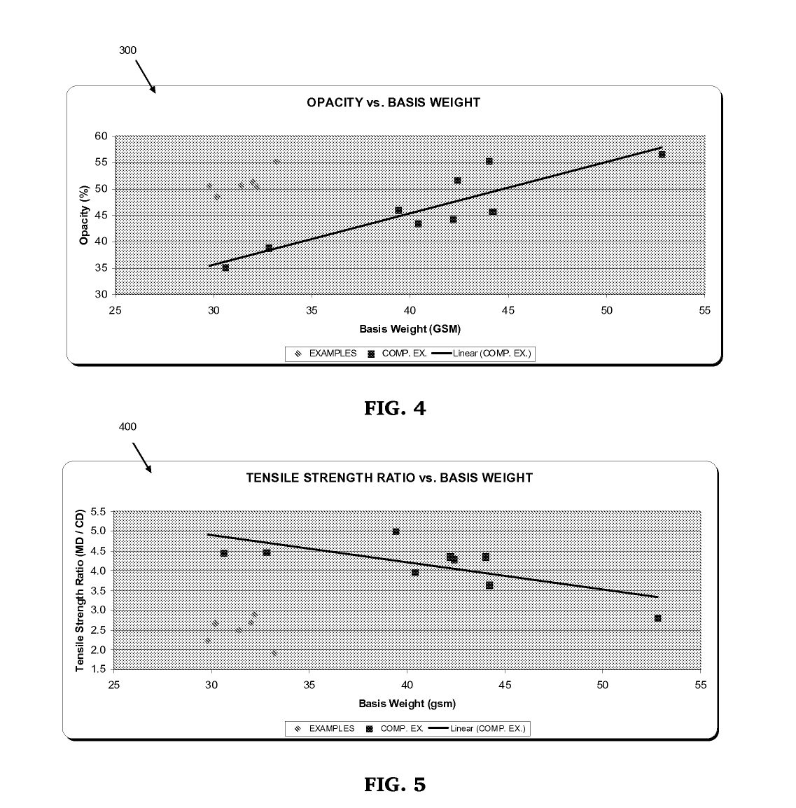

From Table 1, the opacity data is charted against the basis weight data for each of the Examples and Comparative Examples, and the result is shown in the chart provided in FIG. 4, generally designated by reference number 300. Chart 300 shows that Examples 0-5 of the present invention consistently provide higher opacity at lower basis weights as compared to Comparative Examples 1-9, which are standard spunlace products, and a higher opacity at a similar basis weight as compared to Comparative Example 10, which is a composite structure. This illustrates one of the advantages of the present invention, in that a wipe is provided that is relatively light in weight, while still offering the visual security of high opacity. In this regard, an opacity index was calculated for each of the above examples using Equation 1, shown below:

opacity index=(opacity of wipe)/(total basis weight of wipe) (1)

The results of the opacity index calculations for each of the examples are provided in Table 2, shown below:

TABLE 2

| | | | D | E | | |
|---|---|---|---|---|---|---|
| | | C | MD/CD | CD/MD | F | Opacity |
| | | OPACITY | TENSILE | ELONG | BASIS WT | Index |
| | Patent Code | (%) | RATIO | RATIO | (GSM) | (C/F) |
| EXAMPLES | E0 | 50.7 | 2.5 | 1.2 | 31.4 | 1.6 |
| | E1 | 55.1 | 1.9 | 1.2 | 33.2 | 1.7 |
| | E2 | 50.3 | 2.9 | 1.3 | 32.2 | 1.6 |
| | E3 | 51.3 | 2.7 | 1.2 | 32.0 | 1.6 |
| | E4 | 50.6 | 2.2 | 1.1 | 29.8 | 1.7 |
| | E5 | 48.5 | 2.7 | 1.1 | 30.2 | 1.6 |
| COMPARATIVE | CE1 | 35.1 | 4.5 | 3.0 | 30.6 | 1.1 |
| EXAMPLES | CE2 | 38.8 | 4.5 | 3.2 | 32.8 | 1.2 |
| | CE3 | 51.7 | 4.3 | 3.8 | 42.4 | 1.2 |
| | CE4 | 45.7 | 3.6 | 2.5 | 44.2 | 1.0 |
| | CE5 | 43.5 | 4.0 | 2.4 | 40.4 | 1.1 |
| | CE6 | 44.3 | 4.4 | 3.2 | 42.2 | 1.0 |
| | CE7 | 46.0 | 5.0 | 3.9 | 39.4 | 1.2 |
| | CE8 | 55.3 | 4.4 | 4.3 | 44.0 | 1.3 |
| | CE9 | 56.6 | 2.8 | 2.7 | 52.8 | 1.1 |
| | CE10 | 34.6 | 2.2 | 2.6 | 32.0 | 1.1 |

Table 2 shows that Examples 0-5 of the present invention consistently provide a higher opacity index as compared to other wipe products. In particular, the wipe according to various exemplary embodiments of the present invention may have an opacity index of at least 1.3, while the opacity index of other wipe products are typically lower than this value.

Also, from Table 1, the tensile strength ratio data is charted against the basis weight data for each of the Examples and Comparative Examples, and the results are shown in the chart provided in FIG. 5, generally designated by reference number 400. The tensile strength ratio may be defined as the ratio between the tensile strength of the wipe in the machine direction and the tensile strength of the wipe in the cross direction. Chart 400 shows that Examples 0-5 of the present invention consistently provide a lower tensile strength ratio at lower basis weights as compared to Comparative Examples 1-9. A comparable tensile strength ratio was achieved with the composite structure of Comparative Example 10, but as noted above, Comparative Example 10 did not achieve as high an opacity as Examples 1-9. This illustrates another advantage of the present invention, in that a wipe is provided that is relatively light in weight with improved tensile strength characteristics, in that the tensile strength in the cross direction is relatively closer in value to the tensile strength in the machine direction as compared to other wipe structures. In conventional non-woven manufacturing processes, the machine directional strength is typically much greater than the cross directional strength. Unfortunately, the cross directional strength can serve as the “weak link” when it comes to providing adequate fabric strength for the consumer. Also, the consumer appeal of the higher machine directional strength is lessened due to the significantly lower cross direction strengths. The present invention provides a better quality wipe with more uniform multi-directional strength by increasing cross directional strength relative to that achieved in the machine direction.

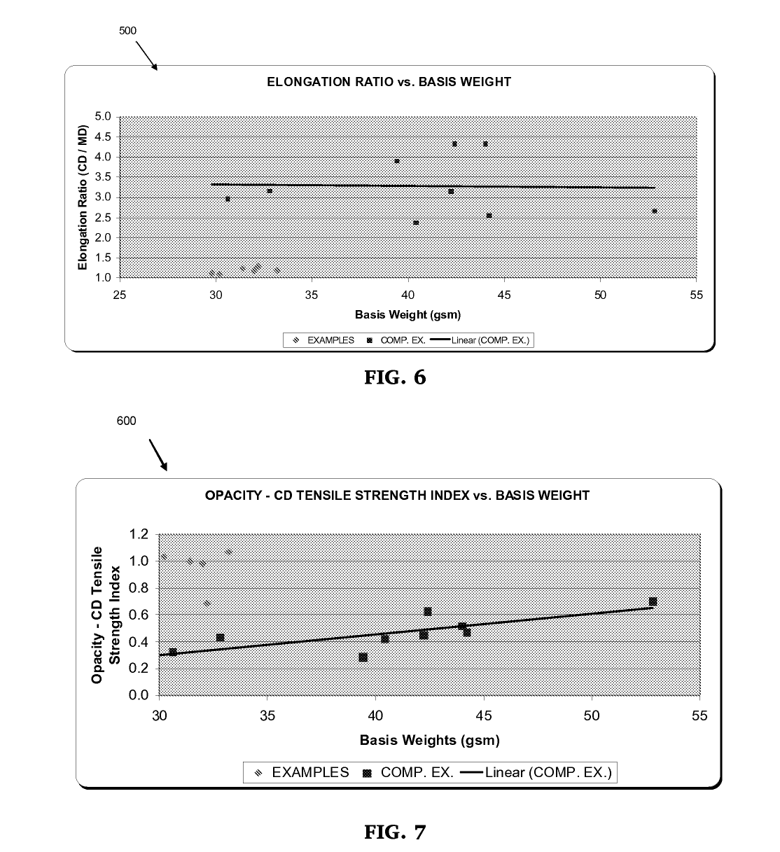

Also, from Table 1, the elongation ratio data is charted against the basis weight data for each of the Examples and Comparative Examples, and the results are shown in the chart provided in FIG. 6, generally designated by reference number 500. The elongation ratio may be defined as the ratio between the percent elongation of the wipe in the cross direction and the percent elongation of the wipe in the machine direction. Chart 500 shows that Examples O-5 of the present invention consistently provide a lower elongation ratio at lower basis weights as compared to Comparative Examples 1-9. This illustrates another advantage of the present invention, in that a wipe is provided that is relatively light in weight with improved elongation characteristics, in that elongation percentage in the cross direction is relatively closer in value to elongation percentage in the machine direction as compared to other wipe structures. In conventional non-woven manufacturing processes, the cross directional elongation is typically much greater than the machine directional elongation. Unfortunately, the cross directional elongation can serve as the “weak link” when it comes to providing adequate wipe integrity for the consumer. In particular, the consumer appeal of a wipe with relatively higher cross directional elongation can be low since the elongation of the fabric can result in significantly different length and width dimensions of the resultant wipe. The present invention provides a better quality wipe with more uniform multi-directional elongation by decreasing the cross directional elongation so as to be closer to the elongation achieved in the machine direction.

In general, the wipe according to various exemplary embodiments of the present invention provides improved quality relative to conventional wipe structures, and in particular is able to provide a combination of increased opacity, increased cross directional tensile strength, and reduced cross directional elongation. In this regard, an opacity-cross dimensional tensile strength index was calculated for each of the above examples using Equation 2, shown below:

opacity-cross dimensional tensile strength index=((opacity of wipe)(cross dimensional tensile strength of wipe))/(total basis weight of wipe)² (2)

The results of the opacity-cross dimensional tensile strength index calculations for each of the examples are provided in Table 3, shown below:

TABLE 3

| | | Opacity - CD |
|---|---|---|
| | | Tensile Strength |
| | Patent | Index |
| | Code | (OPACITY * CDT)/(BW\hat{ }2) |
| EXAMPLES | E0 | 1.0 |
| | E1 | 1.1 |
| | E2 | 0.7 |
| | E3 | 1.0 |
| | E4 | 1.1 |
| | E5 | 1.0 |
| COMPARATIVE | CE1 | 0.3 |
| EXAMPLES | CE2 | 0.4 |
| | CE3 | 0.6 |
| | CE4 | 0.5 |
| | CE5 | 0.4 |
| | CE6 | 0.5 |
| | CE7 | 0.3 |
| | CE8 | 0.5 |
| | CE9 | 0.7 |
| | CE10 | 0.6 |

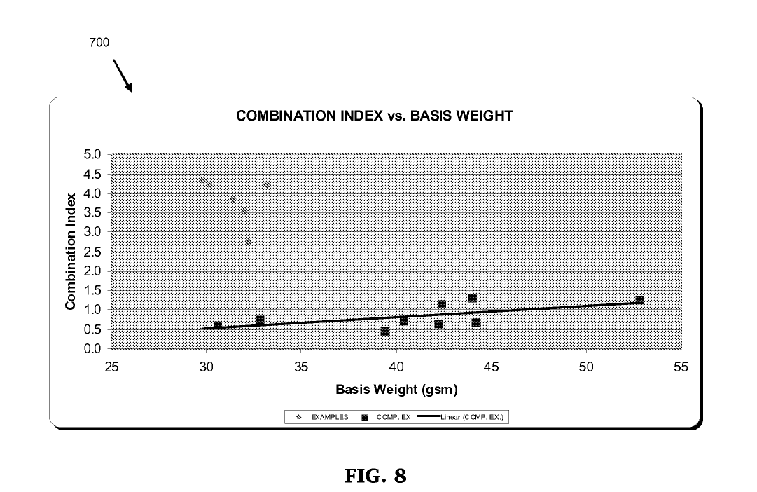

The opacity-cross directional tensile strength index data is charted against basis weight for each of the Examples and Comparative Examples 1-9, and the results are shown in the chart provided in FIG. 7, generally designated by reference number 600. Chart 600 shows that the wipe according to the present invention consistently provides a higher opacity-cross directional tensile strength index (i.e., a combination of both higher opacity and higher cross dimensional tensile strength) as compared to conventional wipe constructions, particularly at low basis weights. In this regard, the wipe according to an exemplary embodiment of the present invention may have an opacity-cross directional tensile strength index of at least 0.5.

In addition, a combination index was calculated for each of the above examples using Equation 3, shown below:

combination index=[((opacity of the wipe)(cross dimensional tensile strength of the wipe)(1/cross dimensional elongation of the wipe))/(total basis weight of the wipe)³](10000) (3)

The results of the combination index calculations for each of the examples are provided in Table 4, shown below:

TABLE 4

| | | COMBINATION |
|---|---|---|
| | | INDEX |
| | | (OP * CDT * (1/CDE))/ |
| | Patent | (BW\hat{ }3) |
| | Code | times 10000 |
| EXAMPLES | E0 | 3.8 |
| | E1 | 4.2 |
| | E2 | 2.7 |
| | E3 | 3.5 |
| | E4 | 4.3 |
| | E5 | 4.2 |
| COMPARATIVE | CE1 | 0.6 |
| EXAMPLES | CE2 | 0.8 |
| | CE3 | 1.2 |
| | CE4 | 0.7 |
| | CE5 | 0.7 |
| | CE6 | 0.6 |
| | CE7 | 0.5 |
| | CE8 | 1.3 |
| | CE9 | 1.2 |
| | CE10 | 1.6 |

The combination index data from Table 4 is charted against basis weight for each of the Examples and Comparative Examples 1-9, and the results are shown in the chart provided in FIG. 8, generally designated by reference number 700. Chart 700 shows that the wipe according to the present invention consistently provides a higher combination index (i.e., a combination of higher opacity, higher cross dimensional tensile strength and lower cross dimensional elongation) as compared to conventional wipe constructions, particularly at low basis weights. In this regard, the wipe according to an exemplary embodiment of the present invention may have a combination index of at least 0.7.

Now that the preferred embodiments of the present invention have been shown and described in detail, various modifications and improvements thereon will become readily apparent to those skilled in the art. Accordingly, the spirit and scope of the present invention is to be construed broadly and limited only by the appended claims and not by the foregoing specification.

1. A wipe comprising:
at least one non-woven web layer of discontinuous fibers; and
a spunbond-meltblown-spunbond web layer of continuous fibers positioned in facing and adjacently contacting relation with the at least one non-woven web layer, wherein the wipe has an opacity index of at least 1.3, where the opacity index is calculated based on the following equation:
opacity index=(opacity of the wipe)/(total basis weight of the wipe).

2. The wipe of claim 1, wherein the at least one non-woven web layer comprises a first non-woven web layer and a second non-woven web layer, the spunbond-meltblown-spunbond web layer disposed between the first and second non-woven web layers.

3. The wipe of claim 1, wherein the discontinuous fibers comprise rayon fibers, natural fibers and polymeric fibers.

4. The wipe of claim 3, wherein the natural fibers comprise at least one of the following natural fiber types: cotton, bamboo, hemp, and pulp.

5. The wipe of claim 3, wherein the polymeric fibers comprise at least one of the following polymeric fiber types: polypropylene, polyester and polylactide.

6. The wipe of claim 1, wherein the at least one non-woven web layer is one of the following types of web layers: carded fiber web layer, air-laid fiber web layer, wet-laid fiber web layer or combinations therof.

7. The wipe of claim 1, wherein the at least one non-woven web layer has a basis of weight within the range of approximately 5 gsm to approximately 55 gsm.

8. The wipe of claim 1, wherein the spunbond-meltblown-spunbond web layer comprises polypropylene.

9. The wipe of claim 1, wherein the spunbond-meltblown-spunbond web layer comprises polylactide.

10. The wipe of claim 1, wherein the spunbond-meltblown-spunbond web layer is unbonded.

11. The wipe of claim 1, wherein the spunbond-meltblown-spunbond web layer is bonded.

12. The wipe of claim 1, wherein the spunbond-meltblown-spunbond web layer has a basis of weight within the range of approximately 5 gsm to approximately 35 gsm.

13. The wipe of claim 1, wherein the at least one non-woven web layer is bonded with the spunbond-meltblown-spunbond material layer.

14. The wipe of claim 1, further comprising a liquid.

15. The wipe of claim 1, wherein the wipe has an opacity-cross dimensional tensile strength index of at least 0.5, where the opacity-cross dimensional tensile strength index is calculated based on the following equation:
opacity-cross dimensional tensile strength index=((opacity of the wipe ) (cross dimensional tensile strength of the wipe))/(total basis weight of the wipe)².

16. The wipe of claim 1, wherein the total basis weight of the wipe is at least 20 gsm.

17. The wipe of claim 1, wherein the opacity of the wipe is at least 40%.

18. The wipe of claim 1, wherein the ratio of tensile strength in the machine direction of the wipe relative to tensile strength in the cross direction of the wipe is within the range of approximately 2.0 to approximately 3.0.

19. The wipe of claim 1, wherein the ratio of percentage elongation in the cross direction of the wipe relative to percentage elongation in the machine direction of the wipe is within the range of approximately 1.0 to approximately 1.5.

20. The wipe of claim 1, wherein the wipe has a combination index of at least 0.7, where the combination index is calculated based on the following equation:
combination index=[((opacity of the wipe)(cross dimensional tensile strength of the wipe)(1/cross dimensional elongation of the wipe))/(total basis weight of the wipe)³](10000).

21. A wipe comprising:
at least one non-woven web layer of discontinuous fibers; and
a spunbond-meltblown-spunbond web layer of continuous fibers positioned in facing and adjacently contacting relation with the at least one non-woven web layer, wherein the wipe has a combination index of at least 0.7, where the combination index is calculated based on the following equation:
combination index=[((opacity of the wipe)(cross dimensional tensile strength of the wipe)(1/cross dimensional elongation of the wipe))/(total basis weight of the wipe)³](10000).

22. A method of forming a wipe, comprising the steps of:
forming at least one non-woven web layer of discontinuous fibers;
forming a spunbond-meltblown-spunbond web layer of continuous fibers; and
bonding the at least one non-woven web layer with the spunbond-meltblown-spunbond web layer, wherein the wipe has an opacity index of at least 1.3, where the opacity index is calculated based on the following equation:
opacity index=(opacity of the wipe)/(total basis weight of the wipe).

23. The method of claim 22, wherein the step of forming the at least one non-woven web layer comprises forming a first non-woven web layer and a second non-woven web layer.

24. The method of claim 23, further comprising disposing the spunbond-meltblown-spunbond web layer between the first and second non-woven web layers.

25. The method of claim 22, wherein the step of forming the at least non-woven web layer comprises using at least one of the following types of web-formation processes: carding, airlaying and wetlaying.

26. The method of claim 22, further comprising bonding the at least one non-woven web layer.

27. The method of claim 26, wherein the step of bonding the at least one non-woven web layer comprises using at least one of the following bonding processes: hydroentanglement, thermal bonding, chemical bonding and mechanical bonding.

28. The method of claim 22, further comprising bonding the spunbond-meltblown-spunbond web layer.

29. The method of claim 22, wherein the step of bonding the at least one non-woven web layer with the spunbond-meltblown-spunbond web layer comprises using at least one of the following bonding processes: hydroentanglement and thermal bonding.

30. The wipe of claim 1, wherein the wipe has an opacity index of at least 1.6.

31. The wipe of claim 1, wherein the wipe has an opacity index within the range of 1.6-1.7.

32. The wipe of claim 1, wherein the wipe has an opacity index of at least 1.7.

33. The wipe of claim 15, wherein the wipe has an opacity-cross dimensional tensile strength index of at least 0.7.

34. The wipe of claim 15, wherein the wipe has an opacity-cross dimensional tensile strength index within the range of 0.7-1.1.

35. The wipe of claim 15, wherein the wipe has an opacity-cross dimensional tensile strength index of at least 1.1.

36. The wipe of claim 20, wherein the wipe has a combination index of at least 2.7.

37. The wipe of claim 20, wherein the wipe has a combination index within the range of 2.7-4.3.

38. The wipe of claim 20, wherein the wipe has a combination index of at least 4.3.

39. The wipe of claim 21, wherein the wipe has a combination index of at least 2.7.

40. The wipe of claim 21, wherein the wipe has a combination index within the range of 2.7-4.3.

41. The wipe of claim 21, wherein the wipe has a combination index of at least 4.3.

42. The wipe of claim 22, wherein the wipe has an opacity index of at least 1.6.

43. The wipe of claim 22, wherein the wipe has an opacity index within the range of 1.6-1.7.

44. The wipe of claim 22, wherein the wipe has an opacity index of at least 1.7.
